(19) United States
(12) Patent Application Publication
(10) Pub. No.: US 2026/0000318 A1
(43) Pub. Date: Jan. 1, 2026

(54) Multi-Modal Activity Parameter Estimation

(71) Applicant: Samsung Electronics Co., Ltd., Suwon-si (KR)

(72) Inventors: Nafiul Rashid, San Jose, CA (US); Ebrahim Nematihosseinabadi, San Jose, CA (US); Mohsin Ahmed, Sunnyvale, CA (US); Jilong Kuang, San Jose, CA (US)

(21) Appl. No.: 19/253,437

(22) Filed: Jun. 27, 2025

Related U.S. Application Data
(60) Provisional application No. 63/666,037, filed on Jun. 28, 2024.

(51) Int. Cl. A61B 5/11; A61B 5/00

(52) U.S. Cl. CPC A61B 5/1118; A61B 5/1123; A61B 5/6898; A61B 5/7275

(57) ABSTRACT

In one embodiment, a method includes determining a set of one or more step-based activity parameters to estimate for a current activity of a user; and determining current parameter-estimation inputs including (1) a current activity context and (2) a current status of each of a number of sensor modalities. The method further includes selecting, for each of the one or more step-based activity parameters and based on the current parameter-estimation inputs, a respective set of one or more sensor modalities from the number of sensor modalities to use to estimate that step-based activity parameter of the current activity of the user; and for each of the one or more step-based activity parameters, estimating that activity parameter using current data from the respective selected set of one or more sensor modalities.

### PRIORITY CLAIM

This application claims the benefit under 35 U.S.C. § 119 of U.S. Provisional Patent Application No. 63/666,037 filed Jun. 28, 2024, which is incorporated by reference herein.

### TECHNICAL FIELD

This application generally relates to multi-modal activity parameter estimation.

### BACKGROUND

Activities such as running have many health benefits. Electronically tracking activities can help a user achieve or maximize health benefits and performance, and can provide the user or a related application (e.g., a heath application) with detailed information about an activity. For example, parameters for step-based activities (e.g., running) include: running cadence, which measure a user's mean steps per minute; vertical oscillation, which measures how much a user's center of mass moves up and down for each step; and ground contact time, which measures how long a user's foot remains on the ground between impact and lift off. These parameters can be used to provide information about a user's running form and running performance. These parameters can also be used to provide information about step-based activities more generally, such as walking, jogging, etc. Other activities (e.g., biking, swimming, etc.) may use other parameters to define the activity or aspects of the activity, and these parameters may be tracked or estimated using an electronic device. For instance, continuing the example in which the activity is running, a user's running cadence may be estimated by motion signals from a wrist-worn device, such as a smartwatch, or by motion signals from a device worn on a waistband of the user.

### BRIEF DESCRIPTION OF THE DRAWINGS

FIG. 1 illustrates an example method for dynamically selecting among available sensor modalities to estimate activity parameters.

FIG. 2 illustrates an example implementation of the example method of FIG. 1 in which a set of one or more devices is selected to estimate each step-based activity parameter.

FIG. 3 illustrates an example implementation in which a parameter selection process and a model selection process are implemented during real-time, step-based activity parameter estimation.

FIG. 4 illustrates an example training process for the run-time process of the example implementation of FIG. 3.

FIG. 5 illustrates an example computing system.

### DESCRIPTION OF EXAMPLE EMBODIMENTS

Electronic devices may be useful for tracking some aspects of an activity, but may be unable or suboptimal for tracking all aspects of an activity. For example, in the context of running, a wrist-worn device such as a smartwatch equipped with an inertial measurement unit (IMU) may accurately detect a user's running cadence, but may provide suboptimal performance for detecting other running parameters such as vertical oscillation or ground contact time. While multiple devices may be paired together, such that the output of the combined devices is used to determine running parameters, the parameter-determining approach is still fixed, in that those multiple devices have to determine the parameters, i.e., there is no option to selectively and dynamically choose among the available devices for determining a particular activity parameter in a particular context. In addition, while using multiple devices can improve activity-parameter determination in certain contexts, doing so often requires a dedicated device that a user otherwise wouldn't have with them. For instance, for a running activity, a device that clips onto a waistband may be used to determine a parameter such as vertical oscillation. However, this device is dedicated to determining specific running parameters, and so a user will have to acquire and wear this dedicated device that the user would not otherwise use.

In contrast, the embodiments of this disclosure provide context-dependent techniques for dynamically selecting the particular sensor modality or modalities, among all available modalities, to use to estimate one or more activity parameters. As explained below, this dynamic selection may be parameter dependent, such that at a given time, a device x may be dynamically selected to determine activity parameter A, while devices x and y may be dynamically selected to collectively determine activity parameter B. In particular embodiments, as illustrated in the preceding example, selecting a particular sensor modality may entail selecting a particular device that embodies that sensor (e.g., selecting a wrist-worn device that includes an IMU). In other embodiments, selecting a particular sensor modality may include selecting among particular sensors on a device. Moreover, as described herein, dynamically selecting the best modality or modalities to use to determine a particular activity parameter allows users to use a variety of sensor modalities on devices that are not dedicated to such purposes and that users typically already use for other purposes, such as smartphones, smartwatches, earbuds, etc., while ensuring that such devices and sensor modalities are only used to determine an activity parameter when doing so provides a superior run-time result for the particular activity context that the user is currently experiencing.

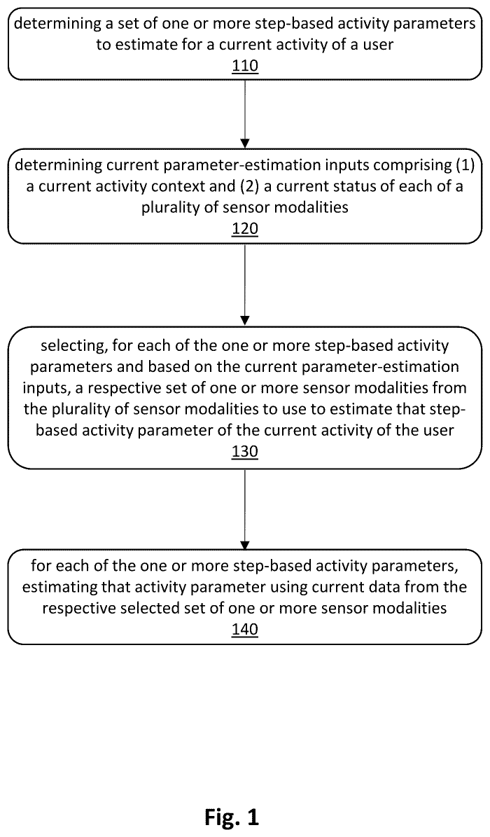

FIG. 1 illustrates an example method for dynamically selecting among available sensor modalities to estimate activity parameters. While the example method of FIG. 1 focuses on step-based activities (e.g., running, walking, jogging, etc.) and corresponding parameters, the techniques described herein may be used to dynamically select the modalities that will be used, in any particular instance, to determine any suitable activity parameters used to characterize or define a particular activity.

Step 110 of the example method of FIG. 1 includes determining a set of one or more step-based activity parameters to estimate for a current activity of a user. For example, the set of step-based activity parameters may include running cadence, vertical oscillation, and/or ground contact time. Other step-based parameters may be used in addition or in the alternative. In particular embodiments, one or more step-based activity parameters may be determined in step 110 of the example method of FIG. 1 by receiving an identification from a user of which parameters to track, or of a specific activity to track parameters for. For example, a user may indicate (e.g., through a health application running on an electronic device of the user, e.g., on a smartphone) which step-based activity parameters the user wishes to track, and this identification may be accessed in step 110. In addition or the alternative, one or more step-based activity parameters may be determined in step 110 of the example method of FIG. 1 by receiving an identification from a software application of which parameters to track or an activity to track. For example, an application related to a rehabilitation therapy for a user may identify particular step-based parameters that are relevant to the user's rehab, and step 110 may include accessing these application-identified parameters.

While the example of FIG. 1 relates to an example in which the activity is a step-based activity, this disclosure contemplates that the techniques described herein may likewise be used for other activities and associated parameters. For example, for a biking activity, parameters related to biking (e.g., pedal cadence, bike speed, etc.) may be determined and estimated using the techniques described herein.

Step 120 of the example method of FIG. 1 includes determining current parameter-estimation inputs that include (1) a current activity context and (2) a current status of each of a number of sensor modalities. The current activity context defines the current activity, from the perspective of accurately estimating the activity parameters identified in step 110, and the labels or classifications of the current activity context correspond to those used during model training, described below. For instance, for a step-based activity, contexts may include walking, jogging, or running, although other contexts may additionally or alternatively be used. The current activity context is the context that corresponds to the user's current activity. The current activity context may be determined by, e.g., user input (e.g., a user may input in a health application that the user is going for a run or for a walk, etc.). In particular embodiments, a current activity context may be determined by an application. For example, an electronic device equipped with an IMU may determine a context-related attribute such as a user's speed, and may estimate the current context (e.g., walking or running) based on the detected attribute(s).

The current status of each of a number of sensor modalities includes whether that current sensor modality is currently available to collect and provide data for parameter estimation. A sensor modality may be a specific sensor, e.g., a specific IMU, microphone, etc. In particular embodiment, a sensor modality may correspond to a particular electronic device. For example, each sensor modality may correspond to a particular, different electronic device (e.g., a smartwatch, a smartphone, a pair of earbuds, etc.), such that determining the current status of a sensor modality is performed by determining the current status of the electronic device that hosts that sensor.

The current status may be detected by, for example, a pairing between a device executing the run-time method of FIG. 1 (e.g., a smartphone) and electronic devices corresponding to the number of sensor modalities. For example, suppose a user is about to go for a run, and the user has their smartphone and smartwatch. The smartwatch may communicate that it is available to the smartphone, indicating that the smartwatch (and therefore constituent sensors, e.g., an IMU of the smartwatch) is currently available. If the user does not have earbuds with them for this run, then the lack of communication from the earbuds may indicate that the earbuds are not available. A current status may be determined automatically (e.g., via communication between devices), manually (e.g., based on user input), or both.

Step 130 of the example method of FIG. 1 includes selecting, for each of the one or more step-based activity parameters and based on the current parameter-estimation inputs, a respective set of one or more sensor modalities from the number of sensor modalities to use to estimate that step-based activity parameter of the current activity of the user. In other words, for each parameter a set of one or more sensor modalities (which may be specific electronic devices, in particular embodiments) are selected in order to provide the optimal context-dependent, parameter-dependent estimation of that parameter.

As explained herein, when multiple devices or sensor modalities are available to estimate an activity parameter, the best combination of device or sensor data to use to estimate that parameter depends on the available sensors (or devices), the context, and can vary based on the particular parameter to be estimated. As one example, if a user has both a smartwatch and a pair of earbuds available to estimate step-based parameters, then for certain parameters (e.g., speed) using output of both devices may result in the best estimation, while for other parameters (e.g., vertical oscillation) using only one device (e.g., earbuds) may result in superior estimation, even though IMU data from both devices is available. As explained below, the specific set of sensor modalities to use for a particular parameter in a particular instance may also depend on a specific model (e.g., regression model) used to estimate the parameters from sensor data. Thus, the best combination of sensors or devices can be selected to make a real-time estimate of a particular activity parameter based on that specific parameter, the current activity context, the available devices, and/or a model to use to estimate the parameter.

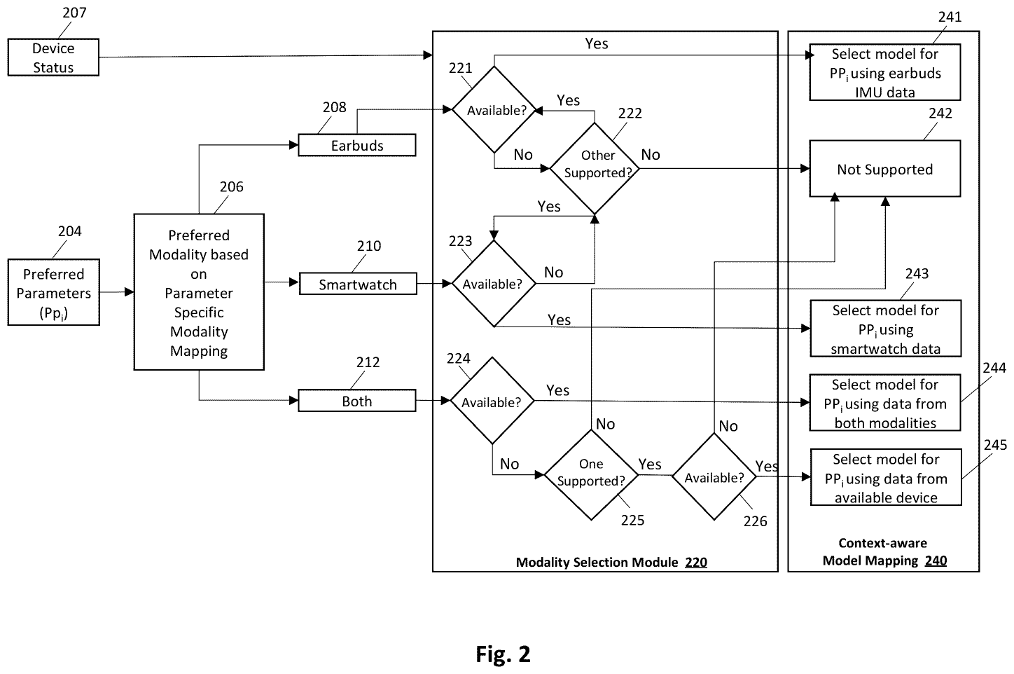

FIG. 2 illustrates an example implementation of step 130 of the example method of FIG. 1 in which a set of one or more devices from a broader set that includes a smartwatch and a pair of earbuds is selected to estimate each step-based activity parameter. In the example of FIG. 2, preferred parameters PPᵢ 204 and device status 202 are obtained, for example as described in connection with steps 110 and 120 of the example method of FIG. 1. For each preferred parameter 204, the preferred set of one or more modalities (here, one or more devices) are identified for that parameter given the current activity context. For example, the optimal set of modalities may be identified from the output of a training phase, described more fully below. In the example, of FIG. 2, for each parameter the set of modalities (devices, in this example) to use to estimate that parameter may be just the earbuds 208, just the smartwatch 210, or both devices 212.

In the example implementation of FIG. 2, once the set of one or more modalities is identified for a particular preferred parameter 204, then modality selection module 220 implements a process to select the modalities to use to estimate that parameter. First, modality selection module determine whether the identified optimal set of one or more modalities is available. For example, if the optimal identified set of modalities is just the earbuds 208, then step 221 identifies whether that particular device is currently available (e.g., based on device status 202). If yes, then in the example of FIG. 2 the process moves to model selection. If not, then step 222 determines whether another modality (here, the smartwatch) is supported to estimate the parameter. In embodiments in which more than two modalities are available, then step 222 may include determining the next optimal (e.g., second-most optimal) set of one or more modalities for estimating the parameter.

If another modality is supported, then modality selection module 220 determines whether that modality or modalities are currently available. For instance, if smartwatch 210 is able to estimate a particular preferred parameter 204, then step 223 may determine whether smartwatch 210 is currently available. If no available modality supports estimating a parameter, then in the example of FIG. 2 the process may end with a “not supported” determination 242, and the user and/or a software application may be notified that no device or sensor is currently available to estimate the parameter.

In the example of FIG. 2, if step 206 identifies both devices 212 as being the optimal set of modalities to estimate a particular preferred parameter 204, then step 224 determines whether both devices are currently available. If not, then step 225 determines whether one of the two devices (in the example of FIG. 2) is able to estimate the parameter on its own. If so, then step 226 determines whether that specific device is available.

The example implementation of FIG. 2 illustrates an embodiment in which, after the set of one or more modalities is selected for a particular parameter, then a particular model to use to estimate that parameter is also identified and selected. In other words, run-time parameter estimation may include dynamically selecting both the modalities to use to estimate that parameter and also a specific estimation model or models to use given the parameter and selected one or more modalities.

For instance, in the implementation of FIG. 2, context-aware model mapping module 240 selects a particular estimation model based on (1) the current activity context (2) the parameter being estimated and (3) the specific selected modality or modalities. For example, if the selected modality in the example of FIG. 2 is only the earbuds 208, then step 241 includes selecting a model to estimate the parameter given that IMU data from the earbuds will be used for that estimation. Likewise, step 243 includes selecting a particular model to use to estimate a parameter when only smartwatch IMU data is used. Steps 244 and 245 select a model to use when data from both devices will be used to estimate the parameter or when only both devices are identified as the optimal set but only one device is available, respectively.

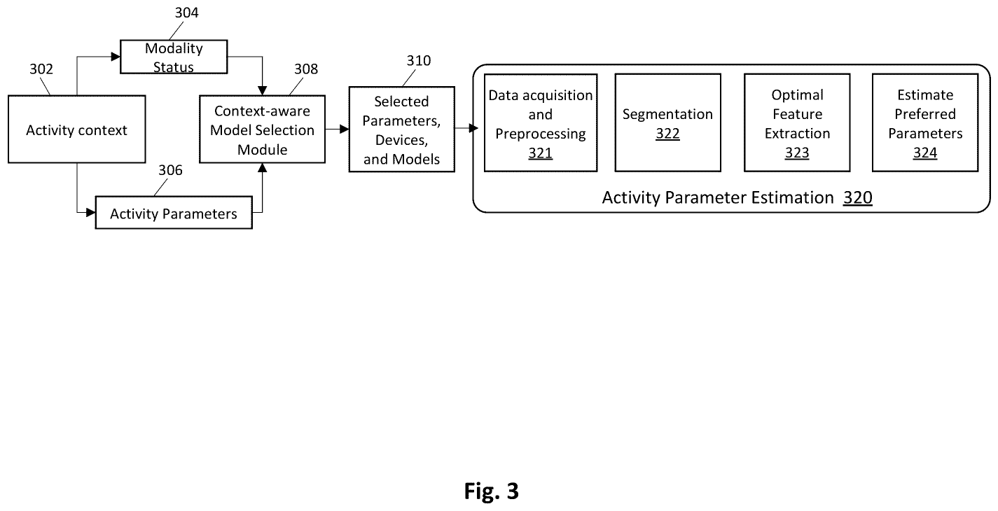

FIG. 3 illustrates an example implementation in which a parameter selection process as well as a model selection process (e.g., the processes illustrated in the example of FIG. 2) are implemented during real-time, step-based activity parameter estimation. In the example of FIG. 3, a step-based context (e.g., running, walking, or jogging, in this example) is determined. Likewise, a modality status (e.g., sensor status or device status) 304 is determined, along with the desired activity parameters 306 to estimate. These inputs are used to make a context-aware modality and model selection 308 (for instance, as illustrated in the example implementation in FIG. 2). The selected parameters, modalities (e.g., devices), and models are then input 310 to activity-parameter estimation process 320.

Step 321 of the example implementation of FIG. 3 includes data acquisition and process 321. Here, data is periodically acquired, e.g., at each sensor's sampling rate. For example, IMU data from a smartwatch may be sampled at a particular rate. For instance, in particular embodiments, the sampling period for a smartwatch may be 20 ms (i.e., 50 Hz). This frequency is sufficient for step-based activities, for instance because the maximum running cadence for a human is typically less than 200 steps per minute or 3.33 steps per second. Parameter estimation for other activities may use other sampling rates that are based on that particular activities' parameters.

Other devices may have a different sampling rate. For example, a pair of earbuds may have an IMU that has a sampling period that varies between 18 to 22 ms. Particular embodiments may package a number of samples (e.g., 20 samples) that includes both accelerometer data (about 3 axes) and gyroscope data (about 3 axes) for each IMU from both the smartwatch and the earbuds and then transmit this data package, e.g., as a burst to the user's smartphone or other connected device, e.g., through Bluetooth. Since the sampling rate for the earbuds in this example is not exactly 50 Hz, then particular embodiments may resample data from both devices to a constant 50 Hz. Particular embodiments may also use spline linear interpolation to fill out gaps in a device's or sensor's data as part of preprocessing step 321.

The example activity parameter estimation process 320 includes segmentation step 322. For example, in this example each IMU's signal amplitude is normalized, e.g., to a −1 to 1 range, and passed through a sliding window. Particular embodiment may use two window sizes of 1 second and 5 seconds (representing intra- and inter-stride variations), with a step size of 1 second, to generate IMU data chunks, although any suitable window size and step size may be used. In this example, the window sizes correspond to the typical running cadence, which is between 150 to 200 steps per minute.

The example activity parameter estimation process 320 includes optimal feature selection step 323. As explained below, particular embodiment during the training phase identify the optimal set of features from all available features to extract from modality data in order to estimate a particular parameter. As explained below, the optimal features may depend on the available modalities that acquired the underlying data, the parameter to be estimated, and/or the activity context. The feature set identified during the training phase is then used during run-time activity parameter estimation to extract the corresponding, optimal set of features from the sensor data.

Finally, step 324 in the example implementation of FIG. 3 estimates the parameter(s) from the selected set of one or more modalities and selected estimation model, which is specific to each particular parameter. Thus, optimized activity parameter estimation is obtained in a parameter-specific, context-dependent manner.

The estimated activity parameters may be used for a variety of purposes. For example, they may be presented to a user, so that the user can track their activity over time. As another example, activity parameters such as step-based activity parameters may be used by one more health applications, such as by a workout-coaching application that provides feedback to a user regarding the user's activity, or by a rehabilitation or therapy application, in order to provide personalized feedback and health monitoring for the user.

While the examples of FIGS. 2-3 illustrate an embodiment in which two devices are available for estimating a parameter, this disclosure contemplates that more than two modalities may be available for selection in the set of modalities for any given parameter. For instance, while certain examples above focus on the use of a smartwatch, earbuds, or combination thereof to estimate step-based activity parameters, any number of devices (e.g., having an IMU sensor that supports step calculations) can be used for step-based activity parameter estimation using the techniques described herein. For example, in an embodiment in which a smartphone is available as a particular modality in addition to earbuds and a smartwatch, then there will be 7 possible combinations of modalities-buds only; watch only; phone only; buds and watch; buds and phone; watch and phone; and buds, phone and watch all together. In general, for N number of modalities (e.g., devices), the total possible combination of modalities will be: C(N,1)+C(N,2)+ . . . C(N,N).

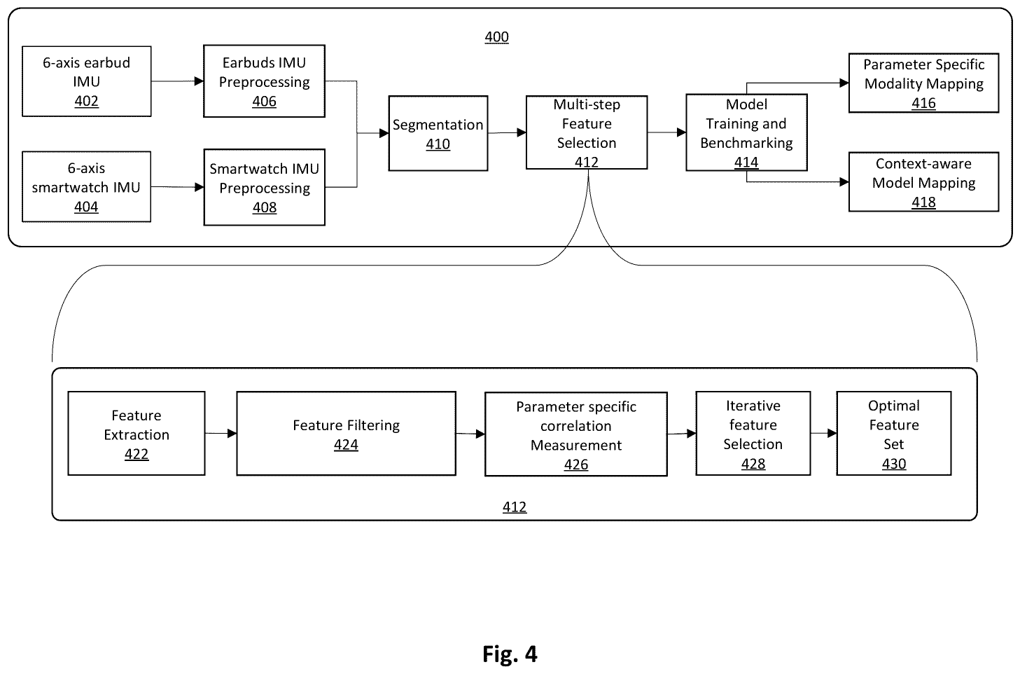

FIG. 4 illustrates an example training process for the run-time process of the example implementation of FIG. 3. In the example of FIG. 4, training data 402 is taken from an IMU in a pair of earbuds, and training data 404 is taken from an IMU of a smartwatch. The IMU data includes data about 6 axes (i.e., acceleration about 3 axes and rotation about 3 axes). The training data is obtained for each context classification that will be used during runtime. For example, training data may be obtained while a user is walking (e.g., for 1 minute), while a user is jogging, and while a user is running, and each set of training data will correspond to that particular activity classification, as described below. In general, training data is obtained from each modality for each context label for a particular activity.

In the example of FIG. 4, preprocessing of earbuds IMU data occurs in step 406, and preprocessing of smartwatch IMU data occurs in step 408. The preprocessing may be as described above with respect to step 321 of FIG. 3.

Step 412 of the example implementation of FIG. 4 includes a multi-step feature selection process. Here, a number of features are extracted (e.g., step 422) from each input channel for each set of training data acquired from each modality. For example, 11 statistical features may be extracted from each of 16 input channels in an embodiment that uses IMU data from earbuds and IMU data from a smartwatch. Here, the 16 channels result from: 3 channels from each device's accelerometer data (so 6 accelerometer channels); 3 channels from each device's gyroscope data; and 4 magnitude channels, each magnitude channel corresponding to the magnitude of the accelerometer signal or the gyroscope signal from a specific device. In particular embodiments, the 11 statistical features may include mean, absolute mean, minimum, maximum, absolute maximum, variance, crest, kurtosis, skewness, range, and interquartile range, although this disclosure contemplates that other statistical features may be extracted from the sensor data.

Using all features (e.g., all 176 features in this example) for parameter estimation may lead to overfitting. Moreover, some features might have repetitive information leading to high collinearity. Thus, a multi-feature selection process may perform a feature filtering process 424, for example by removing features with collinearity more than a particular threshold value (e.g., 0.9). Then, particular embodiments may measure the correlation to the estimation target for each parameter in step 426, and then rank each feature based on that parameter-specific correlation. Next, particular embodiments may perform iterative feature selection 428, for example by performing a sweep of the top features to the whole feature list, adding features one by one to identify the optimal feature set for each parameter. For instance, “optimal” may be determined as the feature set for which where the error is the minimum for test data, although other error metrics may be used. Thus, the optimal feature set 430 may be determined for a particular parameter in a particular activity context. In addition, in particular embodiments a set of optimal features may be determined for each available model used to estimate a parameter; in other embodiments, a feature list may be determined for all available models (i.e., the feature list is the one that performs the best, in aggregate, across the available estimation models for a particular parameter).

Step 414 of the example implementation of FIG. 4 includes model training and benchmarking. Here, after feature selection, the optimal feature set for each parameter and context is sent to a model suite (e.g., a suite of regression models) to find the best model for each scenario. The scenario may depend on the parameter being estimated, the context, and/or on the particular set of modalities used to estimate the parameter. For example, one model may be associated with estimating vertical oscillation during a running context while using only earbud data, while another model may be associated with estimating vertical oscillation during a running context while using both earbud and smartwatch data. In particular embodiment, a suite of regression models to select from may include linear regression (lr), random forest (rf), kernel ridge (kr), gradient boost (gb), svm regression (svr), and xgboost regression (xgb) models, although this disclosure contemplates that any suitable models may be used.

Step 416 of the example training process of FIG. 4 includes parameter-specific modality mapping. For example, based on the performance of different modalities on estimating step-based dynamics parameters, particular embodiments map the best (i.e. lowest error relative to ground-truth data) set of one or more modalities for each parameter. Particular embodiments may measure the performance of the different models in calculating a step-based parameter for a particular context using the different, available sets of one or more modalities to estimate that parameter. Particular embodiments may measure performance using the mean absolute percentage error (MAPE) value of the predicted parameter against ground-truth data. Thus, this module ranks each potential set of modalities for estimating a particular parameter in a particular context. Particular embodiments may output the results as a look-up table of ranked sets of modalities for each activity parameter and for each context. This output may then be used during run-time parameter estimation (e.g., as in step 206 of example implementation of FIG. 2) to select the specific set of one or more modalities best suited to estimate a particular parameter for a particular run-time context.

Step 418 includes performing a context-aware model mapping for each parameter. For instance, this module selects the best model to use for a particular parameter/context/set of modalities combination, for example based on benchmarking results during the training phase. For example, the model with the lowest mean absolute percentage error (MAPE) value relative to the ground truth data may be used to rank model performance. Once the mapping is done during training phase, this mapping will be used by the context-aware model selection module (for example, in context-aware model mapping module 240 of example implementation of FIG. 2) during run-time model selection to select a context-dependent, parameter-dependent model to use to estimate particular parameters.

As discussed herein, while the example of FIGS. 1-4 relate specifically to step-based activity parameters this disclosure contemplates that other activity parameters (e.g., biking, rowing, swimming, weightlifting, etc.) may be optimally estimated from a variety of modalities using the techniques described herein, given the underlying parameters to be estimated.

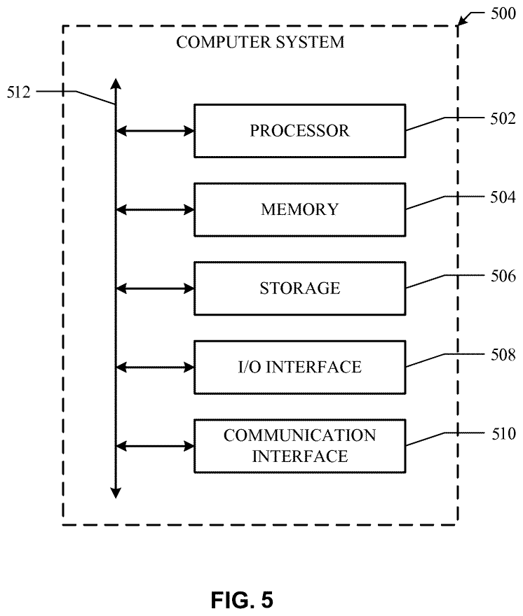

FIG. 5 illustrates an example computer system 500. In particular embodiments, one or more computer systems 500 perform one or more steps of one or more methods described or illustrated herein. In particular embodiments, one or more computer systems 500 provide functionality described or illustrated herein. In particular embodiments, software running on one or more computer systems 500 performs one or more steps of one or more methods described or illustrated herein or provides functionality described or illustrated herein. Particular embodiments include one or more portions of one or more computer systems 500. Herein, reference to a computer system may encompass a computing device, and vice versa, where appropriate. Moreover, reference to a computer system may encompass one or more computer systems, where appropriate.

This disclosure contemplates any suitable number of computer systems 500. This disclosure contemplates computer system 500 taking any suitable physical form. As example and not by way of limitation, computer system 500 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 500 may include one or more computer systems 500; be unitary or distributed; span multiple locations; span multiple machines; span multiple data centers; or reside in a cloud, which may include one or more cloud components in one or more networks. Where appropriate, one or more computer systems 500 may perform without substantial spatial or temporal limitation one or more steps of one or more methods described or illustrated herein. As an example and not by way of limitation, one or more computer systems 500 may perform in real time or in batch mode one or more steps of one or more methods described or illustrated herein. One or more computer systems 500 may perform at different times or at different locations one or more steps of one or more methods described or illustrated herein, where appropriate.

In particular embodiments, computer system 500 includes a processor 502, memory 504, storage 506, an input/output (I/O) interface 508, a communication interface 510, and a bus 512. Although this disclosure describes and illustrates a particular computer system having a particular number of particular components in a particular arrangement, this disclosure contemplates any suitable computer system having any suitable number of any suitable components in any suitable arrangement.

In particular embodiments, processor 502 includes hardware for executing instructions, such as those making up a computer program. As an example and not by way of limitation, to execute instructions, processor 502 may retrieve (or fetch) the instructions from an internal register, an internal cache, memory 504, or storage 506; decode and execute them; and then write one or more results to an internal register, an internal cache, memory 504, or storage 506. In particular embodiments, processor 502 may include one or more internal caches for data, instructions, or addresses. This disclosure contemplates processor 502 including any suitable number of any suitable internal caches, where appropriate. As an example and not by way of limitation, processor 502 may include one or more instruction caches, one or more data caches, and one or more translation lookaside buffers (TLBs). Instructions in the instruction caches may be copies of instructions in memory 504 or storage 506, and the instruction caches may speed up retrieval of those instructions by processor 502. Data in the data caches may be copies of data in memory 504 or storage 506 for instructions executing at processor 502 to operate on; the results of previous instructions executed at processor 502 for access by subsequent instructions executing at processor 502 or for writing to memory 504 or storage 506; or other suitable data. The data caches may speed up read or write operations by processor 502. The TLBs may speed up virtual-address translation for processor 502. In particular embodiments, processor 502 may include one or more internal registers for data, instructions, or addresses. This disclosure contemplates processor 502 including any suitable number of any suitable internal registers, where appropriate. Where appropriate, processor 502 may include one or more arithmetic logic units (ALUs); be a multi-core processor; or include one or more processors 502. Although this disclosure describes and illustrates a particular processor, this disclosure contemplates any suitable processor.

In particular embodiments, memory 504 includes main memory for storing instructions for processor 502 to execute or data for processor 502 to operate on. As an example and not by way of limitation, computer system 500 may load instructions from storage 506 or another source (such as, for example, another computer system 500) to memory 504. Processor 502 may then load the instructions from memory 504 to an internal register or internal cache. To execute the instructions, processor 502 may retrieve the instructions from the internal register or internal cache and decode them. During or after execution of the instructions, processor 502 may write one or more results (which may be intermediate or final results) to the internal register or internal cache. Processor 502 may then write one or more of those results to memory 504. In particular embodiments, processor 502 executes only instructions in one or more internal registers or internal caches or in memory 504 (as opposed to storage 506 or elsewhere) and operates only on data in one or more internal registers or internal caches or in memory 504 (as opposed to storage 506 or elsewhere). One or more memory buses (which may each include an address bus and a data bus) may couple processor 502 to memory 504. Bus 512 may include one or more memory buses, as described below. In particular embodiments, one or more memory management units (MMUs) reside between processor 502 and memory 504 and facilitate accesses to memory 504 requested by processor 502. In particular embodiments, memory 504 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 504 may include one or more memories 504, where appropriate. Although this disclosure describes and illustrates particular memory, this disclosure contemplates any suitable memory.

In particular embodiments, storage 506 includes mass storage for data or instructions. As an example and not by way of limitation, storage 506 may include a hard disk drive (HDD), a floppy disk drive, flash memory, an optical disc, a magneto-optical disc, magnetic tape, or a Universal Serial Bus (USB) drive or a combination of two or more of these. Storage 506 may include removable or non-removable (or fixed) media, where appropriate. Storage 506 may be internal or external to computer system 500, where appropriate. In particular embodiments, storage 506 is non-volatile, solid-state memory. In particular embodiments, storage 506 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 506 taking any suitable physical form. Storage 506 may include one or more storage control units facilitating communication between processor 502 and storage 506, where appropriate. Where appropriate, storage 506 may include one or more storages 506. Although this disclosure describes and illustrates particular storage, this disclosure contemplates any suitable storage.

In particular embodiments, I/O interface 508 includes hardware, software, or both, providing one or more interfaces for communication between computer system 500 and one or more I/O devices. Computer system 500 may include one or more of these I/O devices, where appropriate. One or more of these I/O devices may enable communication between a person and computer system 500. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 508 for them. Where appropriate, I/O interface 508 may include one or more device or software drivers enabling processor 502 to drive one or more of these I/O devices. I/O interface 508 may include one or more I/O interfaces 508, where appropriate. Although this disclosure describes and illustrates a particular I/O interface, this disclosure contemplates any suitable I/O interface.

In particular embodiments, communication interface 510 includes hardware, software, or both providing one or more interfaces for communication (such as, for example, packet-based communication) between computer system 500 and one or more other computer systems 500 or one or more networks. As an example and not by way of limitation, communication interface 510 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 510 for it. As an example and not by way of limitation, computer system 500 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 500 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 500 may include any suitable communication interface 510 for any of these networks, where appropriate. Communication interface 510 may include one or more communication interfaces 510, where appropriate. Although this disclosure describes and illustrates a particular communication interface, this disclosure contemplates any suitable communication interface.

In particular embodiments, bus 512 includes hardware, software, or both coupling components of computer system 500 to each other. As an example and not by way of limitation, bus 512 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 512 may include one or more buses 512, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

This disclosure contemplates a system that includes one or more non-transitory computer readable storage media storing instructions; and one or more processors coupled to the one or more non-transitory computer readable storage media and operable to execute the instructions to perform certain functions includes embodiments in which those functions are performed by a single processor, embodiments in which those functions are performed by multiple processors that each perform all the functions, and embodiments in which those functions are performed by multiple processors (e.g., in separate computing devices) where each processor performs at least one function but less than all recited functions.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
determining a set of one or more step-based activity parameters to estimate for a current activity of a user;
determining current parameter-estimation inputs comprising (1) a current activity context and (2) a current status of each of a plurality of sensor modalities;
selecting, for each of the one or more step-based activity parameters and based on the current parameter-estimation inputs, a respective set of one or more sensor modalities from the plurality of sensor modalities to use to estimate that step-based activity parameter of the current activity of the user; and
for each of the one or more step-based activity parameters, estimating that activity parameter using current data from the respective selected set of one or more sensor modalities.

2. The method of claim 1, wherein selecting the set of one or more sensor modalities comprises selecting a set of one or more electronic devices.

3. The method of claim 2, wherein the current status of each of the plurality of sensor modalities comprises a current status of each of a plurality of electronic devices.

4. The method of claim 2, wherein for each of the one or more step-based activity parameters, estimating that activity parameter comprises:
selecting, based at least on the activity parameter and on the selected set of one or more electronic devices, a particular regression model from a plurality of regression models; and
using the selected particular regression model to estimate the activity parameter from the current data of the respective selected set of one or more electronic devices.

5. The method of claim 2, wherein the set of one or more electronic devices comprises one or more of (1) a smartphone (2) a smartwatch or (3) a pair of earbuds.

6. The method of claim 2, wherein selecting, for each of the one or more step-based activity parameters, the respective set of one or more electronic devices comprises:
determining, for each step-based activity parameter and based on that particular step-based activity parameter and on the current activity context, an optimal set of one or more electronic devices to use to estimate that step-based activity parameter given the current activity context;
determining whether each of the one or more electronic devices in the optimal set is currently available, based on the current status of that respective electronic device;
when each of the one or more electronic devices in the optimal set is currently available, then selecting the optimal set of one or more electronic devices to use to estimate that step-based activity parameter of the current activity of the user; and
when at least one of the one or more electronic devices in the optimal set is not currently available, then selecting a different set of one or more electronic devices to use to estimate that step-based activity parameter of the current activity of the user, based on (1) an accuracy ranking of potential set of electronic devices given the step-based parameter and the current activity context and (2) a current availability of each electronic device.

7. The method of claim 1, wherein determining a set of one or more step-based activity parameters to estimate for a current activity of a user comprises receiving an identification of the one or more step-based activity parameters from one or more of (1) user input or (2) an input from an application running on an electronic device of the user.

8. The method of claim 1, wherein the current activity context comprises an identification of the current activity as one of (1) walking (2) running or (3) jogging.

9. A system comprising:
one or more non-transitory computer readable storage media storing instructions; and one or more processors coupled to the one or more non-transitory computer readable storage media and operable to execute the instructions to:
determine a set of one or more step-based activity parameters to estimate for a current activity of a user;
determine current parameter-estimation inputs comprising (1) a current activity context and (2) a current status of each of a plurality of sensor modalities;
select, for each of the one or more step-based activity parameters and based on the current parameter-estimation inputs, a respective set of one or more sensor modalities from the plurality of sensor modalities to use to estimate that step-based activity parameter of the current activity of the user; and
for each of the one or more step-based activity parameters, estimate that activity parameter using current data from the respective selected set of one or more sensor modalities.

10. The system of claim 9, wherein selecting the set of one or more sensor modalities comprises selecting a set of one or more electronic devices.

11. The system of claim 10, wherein the current status of each of the plurality of sensor modalities comprises a current status of each of a plurality of electronic devices.

12. The system of claim 10, wherein for each of the one or more step-based activity parameters, estimating that activity parameter comprises:
selecting, based at least on the activity parameter and on the selected set of one or more electronic devices, a particular regression model from a plurality of regression models; and
using the selected particular regression model to estimate the activity parameter from the current data of the respective selected set of one or more electronic devices.

13. The system of claim 10, wherein the set of one or more electronic devices comprises one or more of (1) a smartphone (2) a smartwatch or (3) a pair of earbuds.

14. The system of claim 10, wherein selecting, for each of the one or more step-based activity parameters, the respective set of one or more electronic devices comprises:
determining, for each step-based activity parameter and based on that particular step-based activity parameter and on the current activity context, an optimal set of one or more electronic devices to use to estimate that step-based activity parameter given the current activity context;
determining whether each of the one or more electronic devices in the optimal set is currently available, based on the current status of that respective electronic device;
when each of the one or more electronic devices in the optimal set is currently available, then selecting the optimal set of one or more electronic devices to use to estimate that step-based activity parameter of the current activity of the user; and
when at least one of the one or more electronic devices in the optimal set is not currently available, then selecting a different set of one or more electronic devices to use to estimate that step-based activity parameter of the current activity of the user, based on (1) an accuracy ranking of potential set of electronic devices given the step-based parameter and the current activity context and (2) a current availability of each electronic device.

15. A method comprising:
determining a set of one or more activity parameters to estimate for a current activity of a user;
determining current parameter-estimation inputs comprising (1) a current activity context and (2) a current status of each of a plurality of sensor modalities;
selecting, for each of the one or more activity parameters and based on the current parameter-estimation inputs, a respective set of one or more sensor modalities from the plurality of sensor modalities to use to estimate that activity parameter of the current activity of the user; and
for each of the one or more activity parameters, estimating that activity parameter using current data from the respective selected set of one or more sensor modalities.

16. The method of claim 15, wherein selecting the set of one or more sensor modalities comprises selecting a set of one or more electronic devices.

17. The method of claim 16, wherein the current status of each of the plurality of sensor modalities comprises a current status of each of a plurality of electronic devices.

18. The method of claim 16, wherein for each of the one or more activity parameters, estimating that activity parameter comprises:
selecting, based at least on the activity parameter and on the selected set of one or more electronic devices, a particular regression model from a plurality of regression models; and
using the selected particular regression model to estimate the activity parameter from the current data of the respective selected set of one or more electronic devices.

19. The method of claim 16, wherein the set of one or more electronic devices comprises one or more of (1) a smartphone (2) a smartwatch or (3) a pair of earbuds.

20. The method of claim 16, wherein selecting, for each of the one or more activity parameters, the respective set of one or more electronic devices comprises:
determining, for each activity parameter and based on that particular activity parameter and on the current activity context, an optimal set of one or more electronic devices to use to estimate that activity parameter given the current activity context;
determining whether each of the one or more electronic devices in the optimal set is currently available, based on the current status of that respective electronic device;
when each of the one or more electronic devices in the optimal set is currently available, then selecting the optimal set of one or more electronic devices to use to estimate that activity parameter of the current activity of the user; and
when at least one of the one or more electronic devices in the optimal set is not currently available, then selecting a different set of one or more electronic devices to use to estimate that activity parameter of the current activity of the user, based on (1) an accuracy ranking of potential set of electronic devices given the parameter and the current activity context and (2) a current availability of each electronic device.
